(19) United States
(12) Patent Application Publication
(10) Pub. No.: US 2015/0045657 A1
(43) Pub. Date: Feb. 12, 2015

(54) RADIOSCOPY SYSTEM

(71) Applicants: KYUNGPOOK NATIONAL UNIVERSITY INDUSTRY ACADEMIC COOPERATION FOUNDATION, Daegu (KR); KOH YOUNG TECHNOLOGY INC., Seoul (KR)

(72) Inventor: Min-Young Kim, Daegu (KR)

(21) Appl. No.: 14/237,435

(22) PCT Filed: Apr. 19, 2013
(86) PCT No.: PCT/KR2013/003351

§ 371 Date: Feb. 6, 2014

(30) Foreign Application Priority Data
Apr. 27, 2012 (KR) 10-2012-0044778

(51) Int. Cl. A61B 6/00; A61B 19/00; A61B 6/02

(52) U.S. Cl. CPC A61B 6/463; A61B 6/022; A61B 6/4441; A61B 6/4014; A61B 19/54; A61B 2019/5466
USPC 600/424; 378/42

(57) ABSTRACT

A radioscopy system comprises a frame, a radiation generating part, a radiation receiving part, a camera, a central processing part, and a display part. The frame has ring-shaped or partially ring-shaped form. The radiation generating part comprises a first and second radiation generator irradiating toward a first and second surface of an operational object. The radiation receiving part comprises a first and second radiation receiver receiving radiations generated from the first and second radiation generators. The camera comprises a first and second camera capturing a first and second surface of the operational objet. The central processing part generates a first and second fluoroscopy image, a first and second augmented image through combining the first and second fluoroscopy images and the first and second images captured by the first and second cameras. The display part displays the first and second augmented image. Accordingly, doctors may operate the operational object using the augmented images.

### TECHNICAL FIELD

Exemplary embodiments of the present invention relate to a radioscopy system. More particularly, exemplary embodiments of the present invention relate to a radioscopy system for enabling doctors to operate more precisely an operational object.

### BACKGROUND ART

A radioscopy system became widely used for obtaining fluoroscopy images using radiation when examining and operating patient's affected area, and recently, it has been developing surgical method and apparatus using therewith.

Specially, it has been developed a surgical method in recent spinal surgery by capturing image one side of the spinal in 2-D images using C-Arm(unidirectional X-ray), but an operation is performed by fully revealing the affected area, and thus it takes long time for an operation time and recovery time for a patient.

To solve this problem, Korean Patent Registration No. 10-0726022 discloses a surgical measurement system and method used in spinal surgery by using radiation images of both side of the spinal. But, according to this system, a drawback is that doctors may not get a big help to use it properly and apply it to the surgery since it is difficult to determine an exact location of the affected area because only radiation image or fluoroscopy image is used.

Therefore, it is requested to develop a radioscopy system such that a doctor may be provided helpful images and operates more exactly an operational object.

### DISCLOSURE

### Technical Problem

Exemplary embodiments of the present invention provide a radioscopy system capable of generating augmented images such that a doctor may operate more exactly an operational object.

### Technical Solution

According to an embodiment of the present invention, a radioscopy system includes a frame, a radiation generating part, a radiation receiving part, a camera part, a central processing part, and a display part. The frame has ring-shaped or partially-ring shaped form. The radiation generating part is disposed on the frame, and includes a first radiation generator irradiating to a first surface of an operational object and a second radiation generator irradiating to a second surface of the operational object. The radiation receiving includes a first radiation receiver receiving the first radiation that is generated from the first radiation generator and transmits the operational object and a second radiation receiver receiving the second radiation that is generated from the second radiation generator and transmits the operational object. The camera part includes a first camera capturing an image of the first surface of the operational object, and a second camera capturing an image of the second surface of the operational object. The central processing part generates a first fluoroscopy image using the first radiation received from the first radiation receiver, a second fluoroscopy image using the second radiation received from the second radiation receiver, a first augmented image by combining the first captured image generated from the first camera and the first fluoroscopy image, and a second augmented image by combining the second captured image generated from the second camera and the second fluoroscopy image. The display part displays the first and the second augmented images.

In an exemplary embodiment, the radioscopy system may further includes an image matching coordinating part that adjusts at least one of receiving range of the first radiation receiver and a viewing range of the first camera such that the first fluoroscopy image and the first captured image are matched to each other by comparing the first fluoroscopy image from the first radiation receiver and the first captured image from the first camera, images are obtained from a matching reference object that is provided externally before operating an operational object.

In an exemplary embodiment, the radioscopy system may further include a light path converter converting a path of a reflected light such that the reflected light from the first surface by the first camera would be incident.

In an exemplary embodiment, the radioscopy system may further include a radiation generator location adjustor which adjusts a location of the first radiation generator.

In an exemplary embodiment, the radioscopy system may further include a radiation receiver location adjustor which adjusts a location of the first radiation receiver.

In an exemplary embodiment, the radioscopy system may further include a camera adjustor which adjusts a location of the first camera.

In an exemplary embodiment, the radioscopy system may further include a surgical instrument to operate the operational object, the surgical instrument includes a body and a marker for the surgical instrument attached on the body. The radioscopy system may further include a tracking device to detect a location of the marker for the surgical instrument, the tracking device is installed or integrally formed on the first camera or the second camera. In an exemplary embodiment, the radioscopy system may further include a marker attached on the operational object, the tracking device detects the marker for the operational object, and the central processing part matches coordinator systems of the operational object and the surgical instrument by using location information of the operational object detected by the maker and location information of the surgical instrument detected by the maker for surgical instrument.

In an exemplary embodiment, the radioscopy system may further include a shape measurement part that irradiates grating pattern light to the operational object and receives the reflected light from the operational object, and the central processor generates a 3D images using bucket algorithm to the reflected light received from the shape measurement part, and a 3D augmented image using the first and second fluoroscopy images, the first and second images from the first and second cameras, and the generated 3D images.

### Advantageous Effects

According to the present invention, a radioscopy system with a plurality of radiation generators includes cameras apart from a plurality of radiation receivers, and generates augmented images through combining fluoroscopy images and captured images obtained from radiation receivers and cameras, such that doctors may operate more exactly an operational object using the augmented images.

In addition, augmented images may be generated by matching more precisely fluoroscopy images with captured images, and the operational object with the surgical instrument, since all of the coordinators of the operational object such as the surgical instrument, fluoroscopy images, and captured images are matched.

Also, when the radioscopy system includes a shape measurement part for an auxiliary image capturing in addition to the augmented image, it is possible to obtain and display an auxiliary image, and generates 3D augmented image by measuring 3D shape using grating pattern light.

### DESCRIPTION OF DRAWINGS

FIG. 1 is a schematic diagram of a radioscopy system according to an embodiment of the present invention.

FIG. 2 is an augmented images displayed through the radioscopy system according to an embodiment of the present invention of FIG. 1.

FIG. 3 is a schematic diagram explaining coordination system matching between an operation object and a surgical instrument using the radioscopy system of FIG. 1

### MODE FOR INVENTION

The present invention is described more fully hereinafter with reference to the accompanying drawings, in which example embodiments of the present invention are shown. The present invention may, however, be embodied in many different forms and should not be construed as limited to the example embodiments set forth herein. Rather, these example embodiments are provided so that this disclosure will be thorough and complete, and will fully convey the scope of the present invention to those skilled in the art. In the drawings, the sizes and relative sizes of layers and regions may be exaggerated for clarity.

It will be understood that, although the terms first, second, third etc. may be used herein to describe various elements, components, regions, layers and/or sections, these elements, components, and/or sections should not be limited by these terms. These terms are only used to distinguish one element, component, region, layer or section from another region, layer or section. Thus, a first element, component, or section discussed below could be termed a second element, component, or section without departing from the teachings of the present invention.

The terminology used herein is for the purpose of describing particular example embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

For convenience, same numerals are used for identical or similar elements of an apparatus of cutting a tempered substrate and the conventional one.

Hereinafter, with reference to the drawings, it will be described in detail embodiments of the present invention.

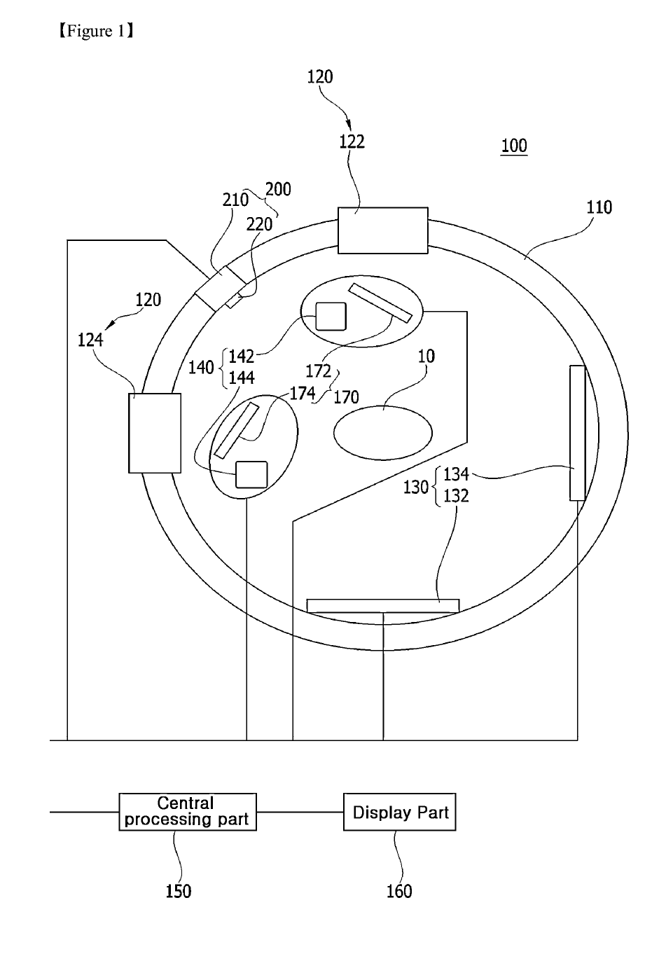

FIG. 1 is a schematic diagram of a radioscopy system according to an embodiment of the present invention.

Referring to FIG. 1, a radioscopy system 100 according to an embodiment of the present invention includes a frame 110, a radiation generating part 120, a radiation receiving part 130, a camera part 140, a central processing part 150 and a display part 160.

The frame 110 may have a ring-shaped or “C” shaped form. Alternatively, the frame 110 may have a partially ring-shaped or “C” shaped form. For example, the frame 110 may be ring-shaped form as shown in FIG. 1, or alternatively, the frame 110 may be partially ring-shaped form, in which a portion of the ring is removed.

The radiation generating part 120 includes at least two radiation generators. The radiation generators are aligned separately on the frame 110, and irradiate radiation toward an operational object 10.

In an exemplary embodiment, the radiation generating part 120 includes a first radiation generator 122 and a second radiation generator 124.

The first radiation generator 122 is disposed on the frame 110 and irradiates a first radiation toward a first surface of the operational object 10. The second radiation generator 124 is disposed separately from the first radiation generator and irradiates a second radiation toward a second surface of the operational object 10.

In an exemplary embodiment, the first radiation generator 122 and the second radiation generator 124 are disposed on the frame 110 in an interval of 90 degrees, each of them may generate and irradiate X-ray toward the operational object 10. The first surface may be an upper surface of the operational object 10 and the second surface may be a left side of the operational object 10. In this case, as showed in FIG. 1, the first radiation generator 122 is disposed on upper side of the frame 110 and irradiates X-ray toward the upper surface of the operational object 10, the second radiation generator 124 is disposed on the left side of the frame 110 and irradiates X-ray toward the left surface of the operational object 10.

The radiation receiving part 130 may include a plurality of receiver corresponding to the radiation generators and receive radiation generated by each of the radiation generators. In an exemplary embodiment, the radiation receiver 130 includes a first radiation receiver 132 and a second radiation receiver 134. The first radiation receiver 132 receives the first radiation that is generated from the first radiation generator 122 and transmitted from the operational object 100 and generated from the first radiation generator 122. The second radiation receiver 133 receives a second radiation transmitted from the operational object 100 and generated from the second radiation generator 124.

In an exemplary embodiment, the first radiation receiver 132 and the second radiation receiver 134 may be disposed on the frame separated in an interval about 90 degrees, about 180 degrees from the first and second radiation generators 122 124, and may receive each transmitted X-rays from the operational object 10. As showed in FIG. 1, the first radiation receiver 132 is disposed on bottom side of the frame 110, the second radiation receiver 134 is disposed in the right side of the frame 110, and receives transmitted X-ray from the operational object 10.

The camera part 140 may include a plurality of camera corresponding to the plurality of radiation receiver. For example, the recorder 140 may include a first camera 142 capturing image of the first surface of the operational object 10 and a second camera 144 capturing image of the second surface of the operational object 10. In FIG. 1, the first camera 142 captures image of the upper surface of the operational object 10 and the second camera captures image of the left surface of the operational object 10. In an exemplary embodiment, the first and the second cameras 142 144 may be selected from CCD camera or CMOS camera.

In an exemplary embodiment, a light source for the camera part 140 may be provided from outside. In other words, it may be selected from lights provided during a surgery such as, natural light, fluorescent light, or incandescent lamp light. Alternatively, the light source for the camera part 140 may be mounted additionally in the radioscopy system 100, or may be used from other light source formed in the radioscopy system 100.

The central processing part 150 generates a first fluoroscopy image using the first radiation received from the first radiation receiver 132, and a second fluoroscopy image using the second radiation received from the second radiation receiver 134. In addition, the central processing part 150 generates a first augmented image through combining the captured image from the first camera 142 and the first fluoroscopy image, and a second augmented image through combining the captured image from the second camera 144 and the second fluoroscopy image. In an exemplary embodiment, the central processing part 150 may be central processing unit of a computing system.

The display part 160 displays the first and second augmented images. The first and second augmented images which are displayed may be applied on the operational object 10 such that a doctor may use them to operate more exactly.

The radioscopy system may further include a light path converter 170. The light path converter 170 may covert path of a reflected light such that a light reflected from small portion of the operational object 10 should be incident to the recorder 140.

In an exemplary embodiment, the light path converting part 170 includes a first light path converter 172 and a second light path converter 174. The first light path converter 172 converts a path of the first reflected light such that the first reflected light from the first surface should be incident to the first camera 142, and the second light path converter 174 converts a path of the second reflected light such that the second reflected light of the second surface should be incident to the second camera 144. For example, each of the first and second light path converters 172 174 may include minors.

Accordingly, by installing the camera part 140 properly to structural features of the radioscopy system 100 and receiving reflected light by the light path converting part 170 in the camera part 140, it is possible to capture exactly images for desired part of the operational object 10.

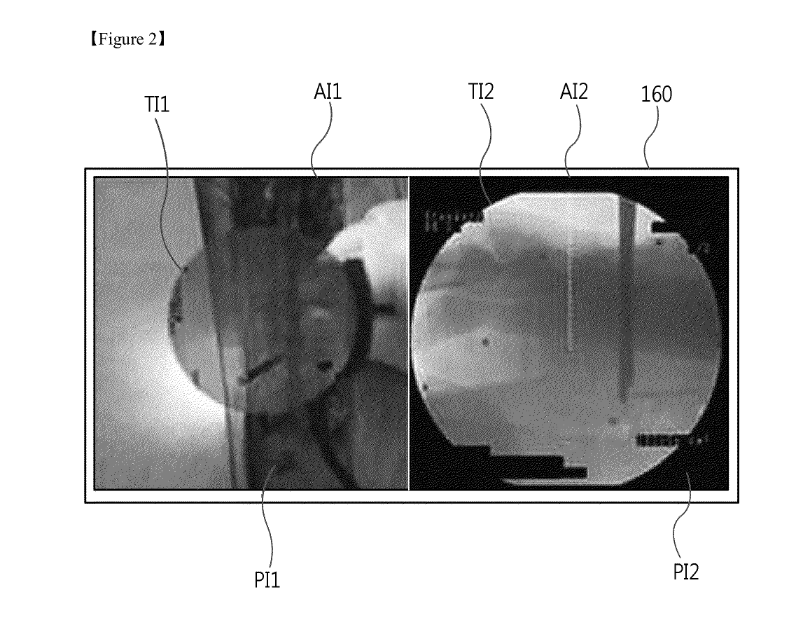

FIG. 2 is an example image of augmented images displayed through the radioscopy system according to an embodiment of the present invention of FIG. 1.

Referring to FIG. 2, the display part 160, includes monitor as an example, the monitor displays a first augmented image AI1 and a second augmented image AI2.

The first augmented image AI1 is an overlapped image between a first fluoroscopy image TI1 and a first captured image PI1, and the second augmented image AI2 is an overlapped image between a second fluoroscopy image TI2 and a second captured image PI2.

Accordingly, doctors may operate more exactly using the first and second augmented images AI1 AI2.

The radioscopy system 100 may further include an image matching coordinating part (not shown).

The image matching coordinating part adjusts at least one of receiving range of the first radiation receiver 132 and a viewing range of the first camera 142 such that the first fluoroscopy image and the first captured image are matched to each other by comparing the first fluoroscopy image from the first radiation receiver 132 and the first captured image from the first camera 142, the images are obtained from a matching reference object that is provided externally before operating the operational object, and adjusts at least one of receiving range of the second radiation receiver 134 and a viewing range of the second camera 144 such that the second fluoroscopy image and the second captured image are matched to each other by comparing the second fluoroscopy image from the second radiation receiver 134 and the second captured image from the second camera 144, the images are obtained from a matching reference object that is provided externally before operating the operational object.

In other words, the image matching coordinating part, before operating the operational object 100, obtains the first fluoroscopy image of the first radiation receiver 132 and the first captured image obtained from the first camera 142 from the matching reference object that is provided externally in advance, and compares the first captured image from the first camera and the first fluoroscopy image of the matching reference object such that the first fluoroscopy image TI1 is matched to the first captured image PI1. Also, the image matching coordinating part, before operating the operational object 100, obtains the second fluoroscopy image of the second radiation receiver 134 and the second captured image obtained from the second camera 144 from the matching reference object that is provided externally in advance, and compares the second captured image from the second camera 144 and the second fluoroscopy image of the matching reference object such that the second fluoroscopy image TI2 is matched to the second captured image PI2.

In continuation, receiving range of the first radiation receiver 132 and viewing range of the first recorder 142 are adjusted such that the first fluoroscopy image and the first captured image from the first camera 142 shows same portion of the operational object 10.

The receiving range of the first radiation receiver 132 may be adjusted by changing at least one of the position of the first radiation generator 122 and the first radiation receiver 142, and the viewing range of the first camera 142 may be adjusted by changing at least one of the position of the first camera 142 and the light path using the first light path converter 172. Also, receiving range of the second radiation receiver 134 and viewing range of the second camera 144 may be adjusted such that the second fluoroscopy image and the second captured image from the second camera 144 shows same portion of the operational object 10. The receiving range of the second radiation receiver 134 may be adjusted by changing at least one of the position of the second radiation generator 124 and the second radiation receiver 144, and the viewing range of the second camera 144 may be adjusted by changing at least one of the position of the second camera 144 and the light path using the second light path converter 174. For example, each of the first and second light path converters 172 74 includes minors, and the viewing range of the first and second cameras 142 144 may be adjusted by changing a tilt angle of the minors.

Accordingly, the image matching coordinating part may include at least one of a first radiation generator location adjustor that adjusts a position of the first radiation generator 122 or a second radiation location adjustor that adjusts a position of the second radiation generator 124. In addition, the image matching coordinating part may include at least one of a first radiation receiver location adjustor that adjusts a position of the first radiation receiver 132 and a second radiation receiver location adjustor that adjusts a position of the second radiation receiver 134. Also, the image matching coordinating part may include at least one of a first camera location adjustor that adjusts a position of the first camera 142 and a second recorder location adjustor that adjusts a position of the second camera 144. Also, the image matching coordinating part may include at least one of the first light path converter 172 and the second light path converter 174.

Meanwhile, as an example, the matching reference may be a plate with a grating pattern, grating point, etc. In this case, for more precise matching, the first radiation generator 122 is aligned toward the matching reference object with plate shape when comparing the first fluoroscopy image and the first captured image from the first camera 142, and the second radiation generator 124 is aligned toward the matching reference object with plate shape when comparing the second fluoroscopy image and the second captured image from the second camera 144.

Therefore, doctor may operate more exactly the operational object 10 by using the first and second augmented images AI1 AI2 as the first and second fluoroscopy images TI1 TI2 are matched with the first and second captured images PI1 PI2 from the first and second cameras respectively more exactly.

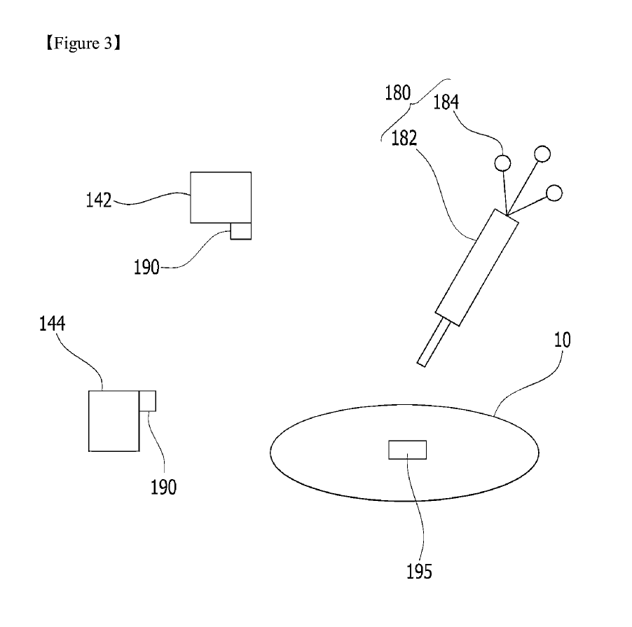

FIG. 3 is a schematic diagram explaining coordination system matching between an operation object and a surgical instrument using the radioscopy system of FIG. 1

Referring to FIG. 3, the radioscopy system 100 may further comprise a surgical instrument 180, a tracking device 190, and a marker 195 for an operational object.

The surgical instrument 180 is used for the operational object 10 in a medical operation, a doctor uses the surgical instrument to operate an affected area of a patient. Meanwhile, the surgical instrument 180 may be installed on an arm of a surgical robot.

The surgical instrument 180 includes a main body 182 and a marker 184 for surgical instrument attached on the main body 182. The marker 184 for surgical instrument is used to communicate with the tracking device 190.

The tracking device 190 detects a location of the marker 184 for surgical instrument. Specifically, the tracking device 190 communicates with the marker 184 for surgical instrument through infrared communication such that the surgical instrument 180 may obtain 3D spatial location information of the surgical instrument 180 in real-time.

The tracking device 190 may be installed on or integrally formed at least one of the first camera 142 and the second camera 144. In FIG. 3, the tracking device 140 is installed on both of the first and second cameras 142 144.

The marker 195 for operational object is attached on the operational object 10. For example, the marker 195 for operational object may be attached on head or small region of a patient. The tracking device 190 detects the marker 195 for operational object. Specifically, the tracking device 190 may obtain 3D spatial location information of the patient by communicating with the marker 195 for operational target through infrared communication.

The central processing part 150 and the surgical instrument use the location information of the operational object 10 detected by the marker 195 and the location information of the surgical instrument 180 detected by the marker 195 to match coordination systems between the operational object 10 with the surgical instrument 180.

Meanwhile, using the first and second fluoroscopy images and the first and second captured images from the first and second cameras 142 144 captured at same time by the operational object 10 and the surgical instrument 180, the surgical instrument 180 or the operational object 10 may match the coordination systems of the first and second fluoroscopy images or the first and second captured images from the first and second cameras 142 144.

As previously described, the coordination systems of the operational object 10, the surgical instrument 180, the fluoroscopy images and the images from the recorders may be matched since the coordination systems of the operational object 100 and the surgical instrument 180 are matched to each other, the coordination systems of each of the first and second fluoroscopy images and the first and second captured images from the first and second cameras 142 144 are matched, and the coordination systems of the surgical instrument 180 or the operational object 10 are matched to the first and second fluoroscopy images or the first and second captured images from the first and second cameras 142 144.

Doctor may operate more exactly the operational object 10 using the first and second augmented images with the coordination systems matched as above.

Referring again to FIG. 1, the radioscopy system may further include a shape measurement part 200.

The shape measurement part 200 is used to obtain auxiliary image for the operational object 10. The shape measurement part 200 simply may obtain a 2D image of the operational object 10 including a camera, but, it also may obtain a 3D image of the operational object with a configuration as described below.

The shape measurement part 200 irradiates grating pattern light toward the operational object and receives reflected grating pattern light from the operational object 10.

The central processing part 150 generates a 3D image applying Bucket algorithm to the received reflected light in the shape measurement part 200, and generates a 3D augmented image using the first and second fluoroscopy images, the first and second captured images from the first and second cameras, and the generated 3D image.

The display part 160 displays the generated 3D image, and doctor may operate more exactly the operational object using the generated 3D augmented image.

In subsequent, the shape measurement of an embodiment the present invention is disclosed.

The shape measurement part 200 includes a projection portion 210 and an image acquisition portion 220.

The projection portion 210 may be located between the first radiation generator 122 and the second radiation generator 124 and aligned about 45 degrees intervals about each of the first and second radiation generator 122 124.

In an exemplary embodiment, the projection portion 210 may include a light source portion, a grating portion, a grating transporting portion and a condensing lens to irradiate the grid pattern light. The light source portion generates light. The grating portion coveters the received light from the light source to the grating pattern light. The grating transporting portion is coupled to the grating portion and transmits the grating portion, for example, PZT (Piezoelectric) transporting portion or any one of the fine line transporting portion may be adopted. The condensing lens is disposed at bottom of the grating portion and irradiates the grating pattern light passing the grating portion to the operational object 10.

In an exemplary embodiment, in the projection portion 210, the grating transporting portion moves the grating portion N times and irradiates a number of N grating pattern lights to the operational object 10, the image acquisition portion 220 receives sequentially the N reflected grating pattern from the operational object 10, and captures images of the pattern image. N is natural number, in an exemplary embodiment, it may be 3 or 4.

The projection portion 210 may use analog pattern scanning device using the PZT transporting portion as described above, or digital pattern scanning device using DMD (digital micro-minor device).

The projection portion may be one or more. When the projection portion is plural, grating pattern lights may be irradiated toward the operational object in a various direction, various type of images are captured, and an error produced from dark shadow region and bright saturation regions caused by a shape of the operational object 10 may be avoided.

The image acquisition portion 220 receives the reflected grating pattern light from the operational object 10 and records the image. In other words, the image acquisition portion 220 receives the reflected grating pattern from the operational body that is generated by the projection portion 210, and captures a planar image of the operational object 10.

The image acquisition portion 220, as referred in FIG. 1, is aligned near the projection portion 210 or integrated in the projection portion 220. Or alternatively, the image acquisition portion 220 may be isolated from the projection portion 210, as an example, it may be placed on an upper region of the operational object 10.

In an exemplary embodiment, the image acquisition portion 220 may include a camera, an imaging lens and a filter. The camera captures planar image of the operational object 10 through receiving reflected light from the operational object 10, as an example, any of CCD camera or CMOS camera may be adopted. The imaging lens is placed at the bottom of the camera, phases the reflected light from the operational body 10 to the camera. The filter is placed at the bottom of the imaging lens, filters out the reflected light from the operational object and provides to the imaging lens, as an example, any of frequency filters, color filter or light intensity adjusting filter may be included.

According to the present invention, the radioscopy system with a plurality of radiation generator includes cameras apart from a plurality of radiation receiver, and generates augmented image through combining fluoroscopy images and captured images obtained from radiation receivers and cameras, such that doctors may operate more exactly an operational object using the augmented image.

In addition, augmented image may be generated by matching more precisely fluoroscopy images with captured images, and the operational object with the surgical instrument, since that, it is possible to match coordinator system of the operational object, the surgical instrument, fluoroscopy images, and recorded images.

Also, when the radioscopy system includes shape measurement part for auxiliary image capturing in addition to an augmented image, it is possible to obtain and display an auxiliary image, and generate a 3D augmented image by measuring a 3D shape using grating pattern light.

It will be apparent to those skilled in the art that various modifications and variation may be made in the present invention without departing from the spirit or scope of the invention. Thus, it is intended that the present invention cover the modifications and variations of this invention provided they come within the scope of the appended claims and their equivalents.

1. A radioscopy system comprising:
a frame having a ring-shaped or partially ring-shaped form;
a radiation generating part part comprising a first radiation generator irradiating a first radiation toward a first surface of an operational object, and a second radiation generator irradiating a second radiation toward a second surface of the operational object;
a radiation receiving part comprising a first radiation receiver receiving the first radiation that is generated from the first radiation generator and transmits the operational object and a second radiation receiver receiving the second radiation that is generated from the second radiation generator and transmits the operational objet;
a camera part comprising a first camera capturing an image of the first surface of the operational object and a second camera capturing an image of the second surface of the operational object;
a central processing part generating a first fluoroscopy image using the first radiation received in the first radiation receiver, a second fluoroscopy image using the second radiation received in the second radiation receiver, a first augmented image combining a first captured image from the first camera and the first fluoroscopy image, and a second augmented image combining a second captured image from the second camera and the second fluoroscopy image; and
a display part displaying the first and the second augmented images.

2. The radioscopy system of claim 1, further comprising:
an image matching coordinating part adjusting at least one of receiving range of the first radiation receiver and a viewing range of the first camera to match the first fluoroscopy image and the first captured-image with each other by comparing the first fluoroscopy image from the first radiation receiver and the first captured mage from the first camera, which are obtained from a matching reference object externally provided, before operating the operational object

3. The radioscopy system of claim 1, further comprising:
a light path convertor converting a path of a reflected light such that the reflected light from the first surface is incident to the first camera.

4. The radioscopy system of claim 1, further comprising:
a radiation generator location adjustor adjusting a location of the first radiation generator.

5. The radioscopy system of claim 1, further comprising:
a radiation receiver location adjustor adjusting a location of the first radiation receiver.

6. The radioscopy system of claim 1, further comprising:
a camera location adjustor adjusting a location of the first camera.

7. The radioscopy system of claim 1, further comprising:
a surgical instrument to operate the operational object,
wherein the surgical instrument comprises a body and a marker for surgical instrument attached on the body.

8. The radioscopy system of claim 7, further comprising:
a tracking device detecting a location of the marker for surgical instrument.

9. The radioscopy system of claim 8, wherein:
the tracking device is installed or integrally formed on at least one of the first and second camera.

10. The radioscopy system of claim 8, further comprising:
a marker for operational target attached on the operational target,
wherein the tracking device detects the marker for operational object, and the central processing part matches coordinator systems of the operational object and the surgical instrument by using location information of the operational object detected by the maker for operational object and location information of the surgical instrument detected by the maker for surgical instrument.

11. The radioscopy system of claim 1, further comprising:
a shape measurement part irradiating a grating pattern light toward the operational object and receiving a reflected light from the operational object, wherein the central processing part generates a 3D images using bucket algorithm to the reflected light received from the shape measurement part and a 3D augmented image using the first and second fluoroscopy images, the first and second captured images, and the generated 3D image.
